(19) United States
(12) Patent Application Publication
(10) Pub. No.: US 2007/0036741 A1
(43) Pub. Date: Feb. 15, 2007

(54) Thickening agents

(75) Inventors: Emmanuel Villard, Saint Christo En Jarez (FR); Lionel Champagnon, Magneux Haute Rive (FR); Fabienne Jehn-Rendu, Saint Just Saint Rambert (FR)

Correspondence Address: HESLIN ROTHENBERG FARLEY & MESITI PC, 5 COLUMBIA CIRCLE, ALBANY, NY, 12203, US

(73) Assignee: SNF SAS, Saint Etienne (FR)

(21) Appl. No.: 11/505,076

(22) Filed: Aug. 16, 2006

Related U.S. Application Data
(63) Continuation-in-part of application No. PCT/FR05/50047, filed on Jan. 27, 2005.

(30) Foreign Application Priority Data
Feb. 17, 2004 (FR) 04.50287

(51) Int. Cl. A61K 8/81

(52) U.S. Cl. 424/70.16

(57) ABSTRACT

Invert water-in-silicone emulsions and a process of production thereof are disclosed. The invert water-in-silicone emulsion is based on polymerizing or copolymerizing one of an ionic, anionic, and/or cationic monomer, or a non-ionic monomer in the presence of a cross-linking agent and transfer agent. The emulsion comprises: a continuous phase having at least one silicone-type oil, and at least two surfactants, at least one of which is a water-in-silicone-type silicone emulsifier and at least one of which is an inversion agent.

### CROSS REFERENCE TO RELATED APPLICATIONS

The application is a continuation-in-part of PCT Application Ser. No. PCT/FR2005/050047 having international filing date of Jan. 27, 2005, published in French as WO 2005/079965 on Sep. 1, 2005, which claims priority from a French patent application 04.50287, filed Feb. 17, 2004. The entire contents of which are incorporated herein by reference.

### FIELD OF THE INVENTION

This invention relates to the field of synthetic polymers, mainly obtained from water-soluble monomers, or mixtures of such monomers, their production method and their applications as thickening agents and/or emulsifiers and/or stabilisers.

### BACKGROUND OF THE INVENTION

More particularly, the invention relates to an invert water-in-silicone emulsion based on cross-linked polymers with ionic, anionic and/or cationic units, including a silicone oil phase, an aqueous phase, at least one silicone-based emulsifier, and at least one oil-in-water (O/W) emulsifying agent, said emulsion containing 10 to 80% by weight of at least one cross-linked anionic, cationic or amphoteric polymer.

By cross-linked polymers, in this invention, we mean polymers resulting from the use of a cross-linking or branching agent during their polymerisation.

The term silicone applies to a vast family of substances which may have properties that differ greatly from one another. In general, silicones are synthetic materials composed of the elements silicon and oxygen combined with organic groups. Depending on the nature of the organic group and the conditions of polymerisation, silicones can be different kinds of substances (liquids, resins, etc.).

We have observed in previous documents:
Patent EP 186361, which presents a thickener in the form of an inverse emulsion. In the description of the invention, it is indicated that at least part of the oil phase of the W/O emulsion may be a silicone oil.
The inverse emulsion also contains a oil-in-water type emulsifier and it is free of inversing agent.
Patent WO 02/44228 related to the preparation of polymers in the form of a liquid dispersion (no or little aqueous phase: water<3%).
Document U.S. Pat. No. 5,216,070 describing a W/O emulsion having a silicone-type emulsifier added in an oil phase free of silicone.
Document JP-03 361854 disclosing a W/O microemulsion rather than an emulsion which, by definition, has a greater particle size. Furthermore, it does not contain any inversion agents.

A very important technical problem for synthetic polymers whose role is to thicken and/or emulsify and/or stabilise aqueous compositions or emulsions consists in finding strong compatibility with the other components used in the final compositions to provide them with optimal stability.

Silicone production is fairly recent, as it started approximately fifty years ago. Developments in research and understanding, however, have made it possible to synthesise new combinations of these polymers, thus further diversifying their potential.

The silicone-based products available in the market today have a wide variety of properties and are used in sectors that are very different from one another. The growing use of silicone compounds, and therefore the increase in synthesised volumes, has significantly brought down the price of these products. From an economic point of view, it is now possible to envisage including them in projects, even those which aim at developing products for which price is an essential parameter.

It is inferred that there is a strong demand for thickening and stabilising silicone-based compositions, for which a solution has not been satisfactorily provided by the previous state of the art.

Given that silicones are both inorganic and organic compounds, their chemical properties reflect this twofold character. For those skilled in the art, this duality will lead, during the preparation of polymer emulsions, to problems of emulsification and problems of stability in the reaction mixture and emulsion after polymerisation.

Thus, to date, no synthetic polymer of the thickening type has been described nor proposed in the form of a water-in-silicone emulsion.

More particularly, the only effective solution that seems to have been found consists in using a dispersion, as presented in patent WO 02/44228, which provides the advantage of not having an aqueous phase, which eliminates the related stability problems. It should be pointed out that, although this patent offers a potential technical solution to the problems encountered by those skilled in the art, it is severely lacking in description, notably not presenting any examples.

The technical problem corresponding to the invention is therefore to propose a water-in-silicone emulsion with thickening and/or emulsifying and/or stabilising capacity.

### SUMMARY OF THE INVENTION

An aspect of the present invention is a “water-in-silicone” inverse emulsion produced by the process of polymerizing or copolymerizing at least one of an ionic monomer, an anionic monomer, a cationic monomer, or a non-ionic monomer in the presence of a cross-linking agent wherein the emulsion comprises:
(a) a continuous phase having at least one silicone-type oil; and
(b) at least two surfactants, at least one of which is a water-in-silicone-type silicone emulsifier and at least one of which is an inversion agent.

A second aspect of the present invention is a method for making a “water-in-silicone” inverse emulsion comprising: providing an aqueous solution having at least one of an ionic monomer, an anionic monomer, a cationic monomer, or a non-ionic monomer, and a cross-linking agent; providing an oil-based solution having at least one silicone-type oil and at least two surfactants, at least one of which is a water-in-silicone-type silicone emulsifier and at least one of which is an inversion agent; and mixing the aqueous solution and the oil-based solution to form the “water-in-silicone” inverse emulsion having at least one of the silicone-type oil in a continuous phase within the inverse emulsion.

### DETAILED DESCRIPTION OF THE INVENTION

Throughout this application, “cross-linked or branched polymer or copolymer” refers to a polymer or copolymer that is cross-linked or branched, obtained by polymerisation in the form of inverse emulsion, well known to those skilled in the art.

By “inversion agent” we mean a surfactant (or a mixture of surfactants) whose effect is to allow phase inversion in the emulsion and dispersion of the polymer in the aqueous medium when used. This generally has an HLB (hydrophilic/lipophilic balance) value that is high enough to obtain oil-in-water emulsions. Generally, the mean HLB value should be greater than 8. Among the principal families of “inversion agents” we can mention: fatty alcohol ethoxylates, fatty acid esters—sorbitan—polyethylene glycols—glycerol, alkyl polyglucosides, etc. Certain silicone compounds such as dimethicone copolyols can also be used.

According to this invention, a polymer-based “water-in-silicone” inverse emulsion has been developed for thickening, emulsifying and/or stabilising aqueous compositions or emulsions with an acidic pH or alkaline pH while providing the final composition obtained with optimum physical characteristics, notably if it also contains silicone compounds.

The emulsion of the invention could be obtained by polymerisation (or, respectively, copolymerisation, together referred to throughout the text and the claims as “polymerisation”) of at least one ionic monomer and possibly other non-ionic monomers, with the presence of at least one cross-linking agent and possibly at least one transfer agent, and presents:
a continuous phase comprising at least one silicone-type oil,
at least 2 surfactants, of which at least one is a silicone emulsifier of the water-in-silicone type and at least one is an inversion agent.

Those skilled in the art, with their own knowledge or through routine tests, will appreciate the degree of the transfer agent and the polymerisation conditions to be used to obtain a final emulsion with the required intrinsic viscosity.

In one advantageous embodiment, the continuous phase comprises 100% by weight of at least one silicone-type oil.

Moreover, it is also possible to concentrate the emulsion using all known techniques, such as azeotropic distillation, for example.

According to a preferred embodiment, the copolymer is obtained using:
10 to 100% mole fraction of at least one monomer with an ionic charge,
0 to 90% mole fraction of at least one monomer with a neutral charge,
the concentration in active matter during polymerisation is preferably between 20 and 50%,
the rate of cross-linking is greater than 10 ppm, preferably greater than 50 ppm, and preferably greater than 200 ppm (considering the MBA) in relation to the polymer or an equivalent cross-linking with a cross-linking agent with different efficacy. Below can be found a non-limited list of monomers which can be used:
   a/ Ionic Monomers:
cationic monomers: of the type dialkylaminoalkyl (meth)acrylate, dialkylaminoalkyl (meth)acrylamide, diallylamine, methyl diallylamine and their quaternary ammonium or acid salts, etc.
anionic monomers: having a carboxylic function (for example, acrylic acid, methacrylic acid, and their salts, etc.), monomers having a sulphonic acid function (for example, 2-acrylamido-2-methyl propane sulphonic acid (AMPS) and their salts, etc.).
   b/ Non-ionic Monomers: acrylamide, methacrylamide, N-vinyl pyrrolidone, vinyl acetate, vinyl alcohol, acrylate esters, allyl alcohol, etc.

It is important to note that, in combination with these monomers, it is also possible to use at least one non-water-soluble monomer such as acrylic, allyl or vinyl monomers with a hydrophobic group.

Below can be found a non-limited list of cross-linking agents: methylene bisacrylamide (MBA), ethylene glycol diacrylate, polyethylene glycol dimethacrylate, diacrylamide, cyanomethyl acrylate, vinyl oxyethyl acrylate, or methacrylate, formaldehyde, triallylamine, glyoxal, glycidyl ether-type compounds such as ethylene glycol diglycidyl ether, or epoxies or any other means known to those skilled in the art to produce cross-linking.

In the rest of the description and in the claims, the term “water-in-silicone-type silicone emulsifying agent” designates a silicone-based surfactant containing a water-soluble part (hydrophilic) and a silicone liquid-soluble part (siliphilic). In practice, it accounts for between 5 and 20% by weight in relation to the emulsion, and preferably between 7 and 15%.

In an advantageous embodiment, the water-in-silicone-type silicone emulsifying agent is non-ionic.

In practice, the water-in-silicone-type silicone emulsifying agent is chosen from the group including dimethicone copolyol, silicone alkanolamides, silicone esters, silicone glycosides, etc.

Below can be found a non-limited list of transfer agents: isopropyl alcohol, sodium hypophosphite, mercaptoethanol, etc. In practice, the transfer agent can be used during polymerisation.

According to another characteristic, the inversion agent accounts for between 2 and 10% by weight in relation to the filler (emulsion+inverter), and preferably between 2.5 and 6% by weight.

Those skilled in the art will be able to choose the best combination based on their own knowledge and the present description, along with the examples that follow.

### EXAMPLES

1/ Polymerization

Each of the polymers described below was obtained by radical polymerisation in inverse emulsion form. An aqueous phase containing the monomer(s) is finely dispersed in a silicone-type oil notably containing at least one silicone-based emulsifying agent. The mixture is then degassed and the polymerisation is initiated in the conventional way.

| | | | | | |
|---|---|---|---|---|---|
| EXAMPLES | TEST 1 | TEST 2 | TEST 3 | TEST 4 | TEST 5 |

AQUEOUS PHASE

| | | | | | | | | | | |
|---|---|---|---|---|---|---|---|---|---|---|
| Monomer(s) | Acrylic acid | 100% mol | Acrylic acid | 100% mol | Acrylic acid | 100% mol | Acrylic acid | 100% mol | Acrylic acid | 100% mol |
| | Sodium salt | | Sodium salt | | Sodium salt | | Sodium salt | | Sodium salt | |
| Cross-linking | MBA | 550 ppm | MBA | 550 ppm | MBA | 550 ppm | MBA | 550 ppm | MBA | 550 ppm |
| agent | (12) | (1) | (12) | (1) | (12) | (1) | (12) | (1) | (12) | (1) |

ORGANIC PHASE

| Oil(s) | CycloPenta | | | | PDMS (2) 10 cst | | | | | |
|---|---|---|---|---|---|---|---|---|---|---|
| | Siloxane (8) | | | | | | | | | |
| Emulsifier(s) or | Sorbitan | 2% | Sorbitan | 4% | Sorbitan | 2% | Sorbitan | 7% | Sorbitan | 10% |
| stabiliser(s) | monooleate | (3) | monooleate | (3) | monooleate | (3) | monooleate | (3) | monooleate | (3) |

| | |
|---|---|
| Aqueous | 70/30 |
| phase/organic | |
| phase ratio | |
| Monomer | 26 |
| concentration | |
| during | |
| polymerisation | |
| % (3): | |
| Observations | UNSTABLE COMPOSITIONS |
| | phase shift in the inverse emulsions even before polymerisation |

| | | | | | |
|---|---|---|---|---|---|
| EXAMPLES | TEST A | TEST B | TEST C | TEST D | TEST E |

AQUEOUS PHASE

| | | | | | | | | | | |
|---|---|---|---|---|---|---|---|---|---|---|
| Monomer(s) | Acrylic acid | 100% mol | Acrylic acid | 100% mol | Acrylic acid | 100% mol | Acrylic acid | 100% mol | Acrylic acid | 100% mol |
| | Sodium salt | | Sodium salt | | Sodium salt | | Sodium salt | | Sodium salt | |
| Cross-linking | MBA | 550 ppm | MBA | 550 ppm | MBA | 550 ppm | MBA | 30 ppm | MBA | 550 ppm |
| agent | (12) | (1) | (12) | (1) | (12) | (1) | (12) | (1) | (12) | (1) |

ORGANIC PHASE

| Oil(s) | PDMS (2) 5 cst | | | | PDMS (2) 10 cst | | PDMS (2) 20 cst | | CycloPenta | |
|---|---|---|---|---|---|---|---|---|---|---|
| | | | | | | | | | Siloxane (8) | |
| Emulsifier(s) or | Silicone | 10% | Silicone | 10% | Silicone | 10% | Silicone | 10% | Silicone | 10% |
| stabiliser(s) | emulsifier | (3) | emulsifier | (3) | emulsifier | (3) | emulsifier | (3) | emulsifier | (3) |
| | No. 1 (4) | | No. 2 (5) | | No. 1 (4) | | No. 1 (4) | | No. 1 (4) | |

| Aqueous | 65/35 | | | | |
|---|---|---|---|---|---|
| phase/organic | | | | | |
| phase ratio | | | | | |
| Monomer | 26 | | | | |
| concentration | | | | | |
| during | | | | | |
| polymerisation | | | | | |
| % (3): | | | | | |
| Observations | Stable inverse emulsions | | | | |
| Viscosity of a | 29000 cps | 31500 cps | 27000 cps | 2000 cps | 30500 cps |
| 1% polymer | | | | | |
| aqueous | | | | | |
| solution (6) | | | | | |

| | | | | | |
|---|---|---|---|---|---|
| EXAMPLES | TEST F | TEST G | TEST H | TEST I | TEST J |

AQUEOUS PHASE

| | | | | | | | | | | |
|---|---|---|---|---|---|---|---|---|---|---|
| Monomer(s) | Acrylic acid | 100% mol | Acrylic acid | 30% mol | Quaternised | 80% mol | APTAC | 100% mol | APTAC | 50% mol |
| | Sodium salt | | AMPS (9) | | MADAME | | (11) | | (11) | |
| | | | Sodium salt | 70% mol | (10) | | | | acrylic acid | 50% mol |
| | | | | | AM | 20% mol | | | sodium salt | |
| Cross-linking | MBA | 800 ppm | MBA (12) | 550 ppm | MBA (12) | 300 ppm | MBA (12) | 600 ppm | MBA (12) | 1000 ppm |
| agent | (12) | (1) | | (1) | | (1) | | (1) | | |

ORGANIC PHASE

| Oil(s) | PDMS (2) 5 cst/ | | PDMS (2) 5 cst | | | | | | | |
|---|---|---|---|---|---|---|---|---|---|---|
| | Isopar J (7) | | | | | | | | | |
| Emulsifier(s) or | Silicone | 10% | Silicone | 10% | Silicone | 10% | Silicone | % | Silicone | 4% |
| stabiliser(s) | emulsifier | (3) | emulsifier | (3) | emulsifier | (3) | emulsifier | (3) | emulsifier | (3) |
| | No. 1 (4) | | No. 1 (4) | | No. 1 (4) | | No. 1 (4) | | No. 1 (4) | |

| Aqueous | 70/30 | | | | |
|---|---|---|---|---|---|
| phase/organic | | | | | |
| phase ratio | | | | | |
| Monomer | 26 | 30 | 40 | 40 | 40 |
| concentration | | | | | |
| during | | | | | |
| polymerisation | | | | | |
| % (3): | | | | | |
| Observations | STABLE inverse emulsions | | | | |
| Viscosity of a | 40000 cps | 20000 cps | 4000 cpa | 5000 cps | 6000 cps |
| 1% polymer | | | | | |
| aqueous | | | | | |
| solution (6) | | | | | |

| | | |
|---|---|---|
| EXAMPLES | TEST K | TEST L |

AQUEOUS PHASE

| | | | | |
|---|---|---|---|---|
| Monomer(s) | NVP | 10% | NVP | 50% |
| | AA NH₄₊ | 90% | AA NH₄₊ | 50% |
| Cross-linking | TAA (13) | 1000 ppm | TAA (13) | 1000 ppm |
| agent | | | | |

ORGANIC PHASE

| Oil(s) | PDMS 5 cst | | PDMS 100% | |
|---|---|---|---|---|
| Emulsifier(s) or | Silicone | 10% | Silicone | 10% |
| stabiliser(s) | emulsifier | (3) | emulsifier | (3) |
| | No. 1 (4) | | No. 1 (4) | |

| Aqueous | 70/30 | 70/30 |
|---|---|---|
| phase/organic | | |
| phase ratio | | |
| Monomer | 26 | 26 |
| concentration | | |
| during | | |
| polymerisation | | |
| % (3): | | |
| Observations | STABLE inverse emulsions | |
| Viscosity of a | 54000 cps | 26000 cps |
| 1% polymer | | |
| aqueous | | |
| solution (6) | | |

Viscosity measurement: viscosity is measured with a Brookfield RVT—20 RPM. The pH is adjusted with a citric acid solution.

For each of tests A to L, an oil-in-water type surfactant (Trideceth 6, for example) was added at concentrations of at least 2% at the end of the process. The effect of this is to allow phase inversion and polymer dispersion in the aqueous medium during use.
(1) ppm (parts per million)/total monomers
(2) PDMS: polydimethylsiloxane—Series DC200 from Dow Corning
(3) %/inverse emulsion mass
(4) Non-ionic silicone surfactant No. 1: DC5225C from Dow Corning—INCI name: cyclopentasiloxane and PEG/PPG-18/18 Dimethicone
(5) Non-ionic silicone surfactant No. 2: DC9011 from Dow Corning—INCI name: cyclopentasiloxane and PEG-12 dimethicone crosspolymer
(6) Measurement with a Brookfield RVT 20 RPM—25° C.
(7) Isopar J: Isoparaffin hydrocarbon
(8) Cyclopentasiloxane—DC245 from Dow Corning
(9) AMPS: 2-acrylamido-2-methyl propane sulphonic acid
(10) MADAME: Dimethylaminoethyl methacrylate
(11) APTAC: acrylamidopropyltrimethyl ammonium chloride
(12) MBA: methylene bisacrylamide
(13) TAA: triallylamine

In view of the aforementioned results, it appears that specific developments are necessary to obtain a polymer in “water-in-silicone” inverse emulsion form.

Indeed, this cannot be obtained under the standard polymerisation conditions as demonstrated by the results of tests 1 to 5. This explains why, to date, no thickening synthetic polymer has been described or proposed in the form of a water-in-silicone emulsion.

According to this invention, it has been discovered that, surprisingly, thanks to an appropriate choice of polymerisation conditions (at least 2 surfactants, one of which is a silicone-based non-standard emulsifier and at least one is an inversion agent), a new family of polymers makes it possible to thicken, emulsify and/or stabilise aqueous compositions or emulsions, whether having acid pH or base pH, while providing the final composition obtained with very high stability and optimum physical characteristics (cf. viscosity measurements), notably if they also contain silicone compounds (very good compatibility).

The examples above demonstrate the diversity of the polymers that can be obtained with this new type of “water-in-silicone” emulsion, for their thickening, emulsifying and/or stabilising properties, and also for their multiple aspects and texture properties This latter is quite essential for cosmetical market. Test K shows that it is possible to manufacture matt siliconized emulsions for not glossy products. Test L improves the stability against UV of the emulsion.

The polymers in emulsion as we have defined them can be incorporated at any temperature. They also provide great flexibility as regards the incorporation stage.

The invention concerns the use of this family of (co)polymers as a thickening and/or emulsifying and/or stabilising agent.

Those skilled in the art will know how to adapt this invention to options or variants not expressly described, without leaving the scope of this invention.

We claim:

1. A “water-in-silicone” inverse emulsion produced by the process of:
polymerizing or copolymerizing at least one of an ionic monomer, an anionic monomer, a cationic monomer, or a non-ionic monomer in the presence of a cross-linking agent wherein the emulsion comprises:
(a) a continuous phase having at least one silicone-type oil; and
(b) at least two surfactants, at least one of which is a water-in-silicone-type silicone emulsifier and at least one of which is an inversion agent.

2. The emulsion as claimed in claim 1, wherein the cationic monomer is chosen from the group consisting of dialkylaminoalkyl (meth)acrylate, dialkylaminoalkyl (meth)acrylamide, diallylamine, methyldiallylamine, and a quaternary ammonium or acid salt thereof.

3. The emulsion as claimed in claim 1, wherein the anionic monomer is chosen from the group consisting of acrylic acid, methacrylic acid, 2-acrylamido-2-methyl propane sulphonic acid, and a salt thereof.

4. The emulsion as claimed in claim 1, wherein the non-anionic monomer is chosen from the group consisting of acrylamide, methacrylamide, N-vinyl pyrrolidone, vinyl acetate, vinyl alcohol, acrylate ester, and allyl alcohol.

5. The emulsion as claimed in claim 1, wherein the non-ionic monomer is N-vinyl pyrrolidone.

6. The emulsion as claimed in claim 1, wherein the cross-linking agent is chosen from the group consisting of methylene bisacrylamide, ethylene glycol diacrylate, polyethylene glycol dimethacrylate, diacrylamide, cyanomethyl acrylate, vinyl oxyethyl (meth)acrylate, formaldehyde, glyoxal, glycidyl ether and epoxy.

7. The emulsion as claimed in claim 1 additionally comprising a transfer agent.

8. The emulsion as claimed in claim 7, wherein the transfer agent is chosen from the group consisting of isopropyl alcohol, sodium hypophosphite, and mercaptoethanol.

9. The emulsion as claimed in claim 1, wherein the concentration of active matter during polymerization is from 20% by weight (wt %) to 50 wt %.

10. The emulsion as claimed in claimed 1, wherein the water-in-silicone-type silicone emulsifier is non-ionic.

11. The emulsion as claimed in claim 1, wherein the inversion agent has a mean hydrophilic/lipophilic balance (HLB) value greater than 8 and is chosen from the group consisting of a fatty alcohol ethoxylate, a fatty acid ester, sorbitan, a polyethylene glycol, glycerol, alkyl polyglycoside, and a dimethicone copolyol.

12. The emulsion as claimed in claim 1, wherein a cross-linking rate of polymerization or copolymerization is greater than 10 ppm in relation to a polymer formed when the cross-linking agent is methylene bisacrylamide.

13. The emulsion as claimed in claim 1, wherein polymerization or copolymerization occurs in the presence of at least one non-water-soluble monomer selected from the group consisting of an acrylic monomer, allylic monomer, and a vinylic monomer each having a hydrophobic substituent attached.

14. The emulsion as claimed in claim 1, wherein the water-in-silicone-type silicone emulsifier is chosen from the group consisting of a dimethicone copolyol, a silicone alkanolamide, a silicone ester, and a silicone glycoside.

15. The emulsion as claimed in 1, wherein a cross-linking rate of polymerization or copolymerization is greater than 50 ppm in relation to a polymer formed when the cross-linking agent is methylene bisacrylamide.

16. The emulsion as claimed in claim 1, wherein a cross-linking rate of polymerization or copolymerization is greater than 200 ppm in relation to a polymer formed when the cross-linking agent is methylene bisacrylamide.

17. A method for making a “water-in-silicone” inverse emulsion comprising:
providing an aqueous solution having at least one of an ionic monomer, an anionic monomer, a cationic monomer, or a non-ionic monomer, and a cross-linking agent;
providing an oil-based solution having at least one silicone-type oil and at least two surfactants, at least one of which is a water-in-silicone-type silicone emulsifier and at least one of which is an inversion agent; and
mixing the aqueous solution and the oil-based solution to form the “water-in-silicone” inverse emulsion having at least one of the silicone-type oil in a continuous phase within the inverse emulsion.
